(19) United States
(12) Patent Application Publication
(10) Pub. No.: US 2011/0004003 A1
(43) Pub. Date: Jan. 6, 2011

(54) Compound, Photochromic Material, Electronic Material, Compound Producing Method, 2,3-BIS(N,N-BIS(P-ANISYL)4-Minophenylethynyl)Dimethylfumarate Producing Method and 2,3-BIS(N,N-BIS(P-ANISYL)4-Aminophenylethynyl)Dimethylmaleate Producing method

(75) Inventors: Hiroshi Nishihara, Bunkyo-ku (JP); Ryota Sakamoto, Bunkyo-ku (JP)

Correspondence Address: GARCIA-ZAMOR INTELLECTUAL PROPERTY LAW;ATTN: RUY GARCIA-ZAMOR, 12960 LINDEN CHURCH ROAD, CLARKSVILLE, MD, 21029, US

(73) Assignee: The University of Tokyo, Tokyo (JP)

(21) Appl. No.: 12/865,460

(22) PCT Filed: Aug. 1, 2008
(86) PCT No.: PCT/JP2008/063917

§ 371 Date: Jul. 30, 2010

(30) Foreign Application Priority Data
Feb. 6, 2008 (JP) 2008-027090

(51) Int. Cl. C07C 229/44; C07D 209/82

(52) U.S. Cl. 548/440; 560/44; 204/157.82; 204/157.71

(57) ABSTRACT

A novel high-performance material is provided. A certain aspect of the present invention lies in a 2,3-bis(N,N-bis(p-anisyl)4-aminophenylethynyl)dimethylfumarate producing method, characterized by illuminating light onto 2,3-bis(N,N-bis(p-anisyl)4-aminophenylethynyl)dimethylmaleate. Also, a further certain aspect of the present invention lies in a 2,3-bis(N,N-bis(p-anisyl)4-aminophenylethynyl)dimethylmaleate producing method, characterized by illuminating light onto 2,3-bis(N,N-bis(p-anisyl)4-aminophenylethynyl)dimethylfumarate. According to this constitution, there can be obtained a high-performance material by a simple technique.

### TECHNICAL FIELD

The present invention relates to a compound and particularly to a photochromic material.

### BACKGROUND ART

An organic EL (Organic Electro-Luminescence) belongs to a technology which is expected to be applied to a thin display, an illumination or the like. Presently, the technology thereof is used also in mobile equipments such as a mobile phone and the like and hereafter, an application thereof to a next candidate display as an alternative to a thin television (liquid crystal, plasma display, etc.) is particularly expected. The market size of the organic EL is anticipated to exceed the money amount from several hundreds billion yen to one trillion yen, and there has been promoted the development thereof actively toward the practical use centering around the chemical companies, home electronics companies and printing companies in Japan, Korea and Germany.

Triarylamine-based molecules are hole-transport materials having high characteristics and belong to a material group which is used most frequently as a hole transport layer for an organic EL.

By using this molecule, several attempts for synthesizing a light-electronic material having a further higher function have been carried out. For example, by incorporating the molecule into an arylenevinylene-based pai-conjugated polymer, a high-mobility material for an organic TFT (Thin Film Transistor) is synthesized and by combining the molecule with fullerene which is an electron acceptor, a photoelectric conversion material is synthesized.

However, a triarylamine-based material having a function or a material property other than that is rare and there has been expected a development of a novel high-performance light-electronic material which takes advantage of the feature of this molecule.

### DISCLOSURE OF THE INVENTION

### Problem to be Solved by the Invention

The present invention is invented in view of the background technology mentioned above and has an object to provide a novel high-performance material.

### Means for Solving the Problem

According to this invention, in order to achieve the object mentioned above, there are employed constitutions just as described in the patent claims. Hereinafter, this invention will be described in detail.

A first aspect of the present invention lies in a compound, characterized by being expressed by a general formula (1) mentioned below or by a general formula (2) mentioned below.

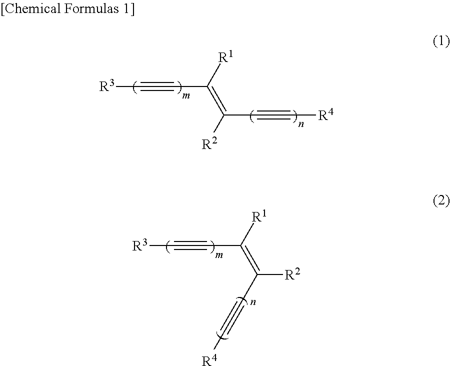

[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an 8-alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group; an aryloxymethyl group; an arylethynyl group; a 4-aryl-buta-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

According to this constitution, there can be obtained a high-performance material in which isomerization by means of an electromagnetic wave is possible.

It should be noted here that it is allowed for the aryl group to be, for example, a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group; or a derivative of one of those groups.

A second aspect of the present invention lies in the compound according to claim 1, characterized in that each of aforesaid R³ and aforesaid R⁴ is identical or different for every appearance thereof and is a group expressed by a general formula (3) mentioned below.

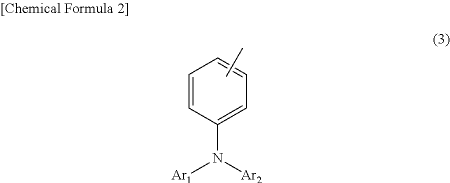

[In the formula, each of Ar¹ and Ar² is identical or different for every appearance thereof and indicates an aryl group having C atoms between 6 or more and 40 or less; a heteroaryl group having C atoms between 2 or more and 40 or less; an aryl group substituted by 1 or more R⁵ groups and having C atoms between 6 or more and 40 or less; or a heteroaryl group having C atoms between 2 or more and 40 or less. R⁵ is identical or different for every appearance thereof and indicates a linear, branched or annular alkyl group having H, F, Cl, Br, I, CN, NO₂, OH, Si(R⁵)₃, N(R⁶)₂, B(R⁵)₂ and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine); or an aryl group having C atoms between 6 or more and 40 or less; a heteroaryl group having C atoms between 2 or more and 40 or less; an aryloxy group having C atoms between 6 or more and 40 or less or a heteroaryloxy group having C atoms between 2 or more and 40 or less (it is allowed for each one of those groups to be substituted by a linear, branched or annular alkyl group having 1 or more of H, F, Cl, Br, I, CN, NO₂, OH, Si(R⁶)₃, N(R⁶)₂ and B(R⁶)₂, and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine). It is allowed for two or more R⁵ groups to mutually form monocyclic or polycyclic cyclic systems of fatty series or aromatic series. R⁶ is identical or different for every appearance thereof and indicates a hydrogen, aliphatic or aromatic hydrocarbon group having H and C atoms between 1 or more and 20 or less.]

According to this constitution, there can be obtained a high-performance material in which isomerization by means of an electromagnetic wave is possible.

A third aspect of the present invention lies in the compound according to claim 1, characterized in that each of aforesaid R³ and aforesaid R⁴ is identical or different for every appearance thereof and is a group expressed by a general formula (4) mentioned below.

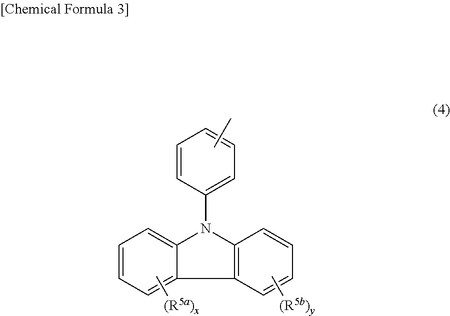

[In the formula, each of x and y is an integer value between 0 or more and 4 or less. Each of R^{5a} and R^{5b} is identical or different for every appearance thereof and indicates a linear, branched or annular alkyl group having H, F, Cl, Br, I, CN, NO₂, OH, Si(R⁶)₃, N(R⁶)₂, B(R⁶)₂ and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine); an aryl group having C atoms between 6 or more and 40 or less; a heteroaryl group having C atoms between 2 or more and 40 or less; an aryloxy group having C atoms between 6 or more and 40 or less; or a heteroaryloxy group having C atoms between 2 or more and 40 or less (it is allowed for each one of those groups to be substituted by a linear, branched or annular alkyl group having 1 or more of H, F, Cl, Br, I, CN, NO₂, OH, Si(R⁶)₃, N(R⁶)₂ and B(R⁶)₂, and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine). It is allowed to form a monocyclic or polycyclic cyclic system of fatty series or aromatic series among two or more of R^{5a} group comrades or R^{5b} group comrades, or between a R^{5a} group and a R^{5b} group. R⁶ is identical or different for every appearance thereof and indicates an aliphatic or aromatic hydrocarbon group having H and C atoms between 1 or more and 20 or less.]

According to this constitution, there can be obtained a high-performance material in which isomerization by means of an electromagnetic wave is possible.

A fourth aspect of the present invention lies in a compound, characterized by being expressed by a formula (5) mentioned below or by a formula (6) mentioned below.

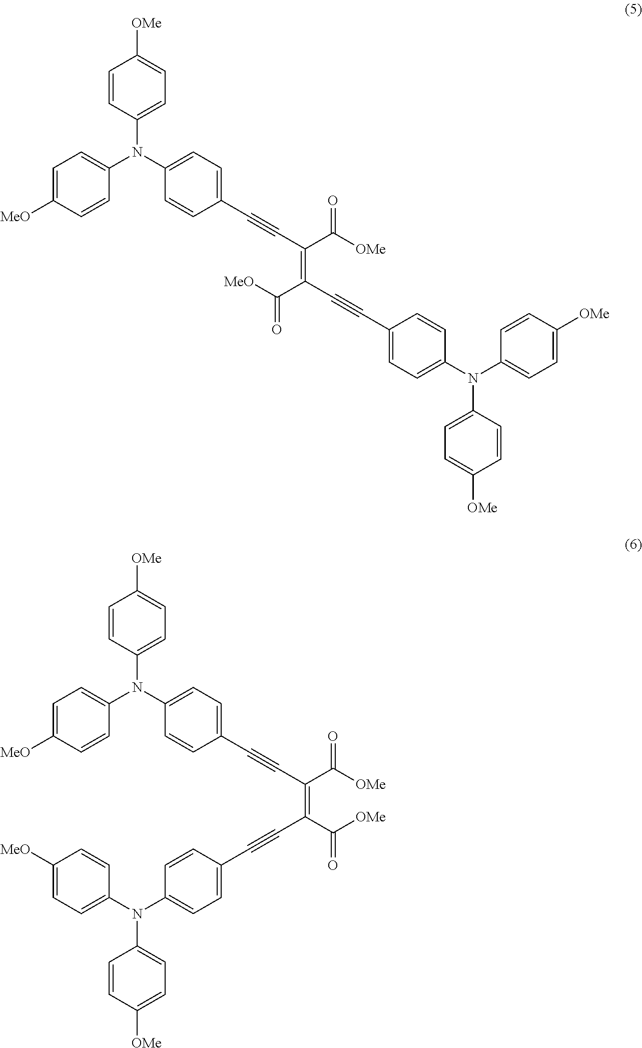

According to this constitution, there can be obtained a high-performance material in which isomerization by means of an electromagnetic wave is possible.

A fifth aspect of the present invention lies in a photochromic material, characterized by using the compound according to claim 1 as the material thereof.

A sixth aspect of the present invention lies in an electronic material, characterized by using the compound according to claim 1 as the material thereof.

A seventh aspect the present invention lies in a compound producing method, characterized by producing a compound expressed by a general formula (2) mentioned below by illuminating an electromagnetic wave onto a compound expressed by a general formula (1) mentioned below.

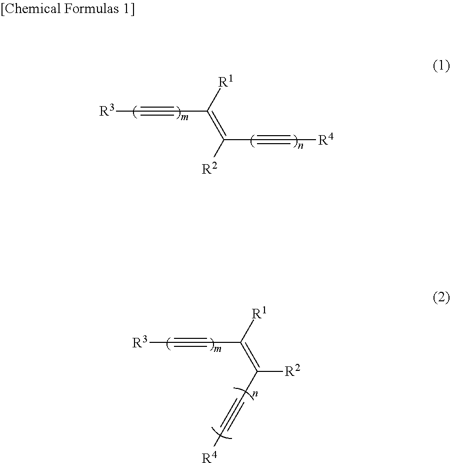

[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group; an aryloxymethyl group; an arylethynyl group; a 4-aryl-octa-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

According to this constitution, there can be obtained a high-performance material by a simple technique.

It should be noted here that it is allowed for the aryl group to be, for example, a ferrocenyl group; an N,N-diaryl-9-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group; or a derivative of one of those groups.

An eighth aspect of the present invention lies in a compound producing method, characterized by producing a compound expressed by a general formula (1) mentioned below by illuminating an electromagnetic wave onto a compound expressed by a general formula (2) mentioned below.

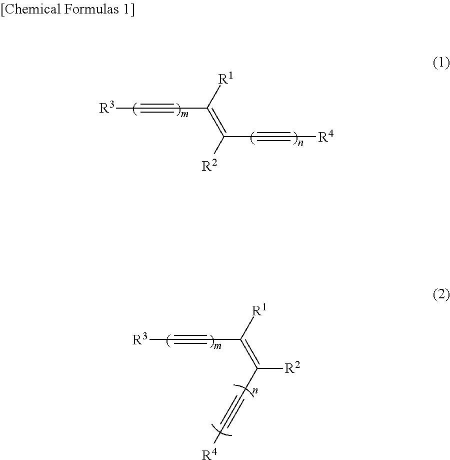

[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an 8-alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group; an aryloxymethyl group; an arylethynyl group; a 4-aryl-buta-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

According to this constitution, there can be obtained a high-performance material by a simple technique.

It should be noted here that it is allowed for the aryl group to be, for example, a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group; or a derivative of one of those groups.

A ninth aspect of the present invention lies in a 2,3-bis(N,N-bis(p-anisyl)-4-aminophenylethynyl)dimethylfumarate producing method, characterized in that a 2,3-dibromo-dimethylfumarate and an N,N-bis(p-anisyl)-4-ethynyl-benzenamin are reacted.

According to this constitution, there can be obtained a high-performance material by a simple technique.

A tenth aspect of the present invention lies in a 2,3-bis(N,N-bis(p-anisyl)-4-aminophenylethynyl)dimethylfumarate producing method, characterized in that light is illuminated onto a 2,3-bis(N,N-bis(p-anisyl)-4-aminophenylethynyl)dimethylmaleate.

According to this constitution, there can be obtained a high-performance material by a simple technique.

An eleventh aspect of the present invention lies in a 2,3-bis(N,N-bis(p-anisyl)-4-aminophenylethynyl)dimethylmaleate producing method, characterized in that light is illuminated onto a 2,3-bis(N,N-bis(p-anisyl)-4-aminophenylethynyl)dimethylfumarate.

According to this constitution, there can be obtained a high-performance material by a simple technique.

### EFFECT OF THE INVENTION

According to this constitution, there can be obtained high-performance material in which isomerization by means of an electromagnetic wave is possible or the like.

Still other objects, features or advantages of the present invention will become clear according to exemplified embodiments of the present invention and detailed explanations based on the attached drawings described later.

### BRIEF DESCRIPTION OF DRAWINGS

FIG. 1A is a diagram showing cyclic voltammograms of (a): a compound (E)-1 and (b): a compound (Z)-1;

FIG. 1B is a diagram showing (a): cyclic voltammograms of (E)-2, (Z)-2 and (b): a simulation with respect to the voltammogram of (E)-2;

FIG. 2 is a diagram showing UV-vis spectra of the compounds (E)-1 and (Z)-1;

FIG. 3 is an ORTEP diagram of the compound (E)-1;

FIG. 4 is a drawing showing IR spectra originated from C═O stretching vibrations of the compounds (E)-1, (Z)-1, (E)-2 and (Z)-2;

FIG. 5A is a diagram showing (a): a UV-vis spectral change (toluene solution) and (b): ¹H-NMR spectral change (toluene-d₈ solution) both associated with visible light illumination with respect to the compound (E)-1;

FIG. 5B is a diagram showing (a) & (b): moment-to-moment change of the associated UV-vis spectrum with respect to the compound (E)-1 when illuminating a visible light (405 nm) thereon and (c): a plot of quantum-yield calculation and a used parameter;

FIG. 5C is a diagram showing (a): a UV-vis spectral change of the (E)-2 in the inside of CH₂Cl₂ and (b): a ¹H-NMR spectral change of the (E)-2 in the inside of CD₂Cl₂ & ¹H-NMR spectrum of the (Z)-2 both associated with a visible light (546 nm, 578 nm) illumination;

FIG. 5D is a diagram showing a reversible switch of an electronic coupling between triarylamines by a visible light in the (E)-1;

FIG. 5E is a diagram showing a switch of an electronic coupling between ferrocenes by a visible light in the (E)-2;

FIG. 6 is a diagram showing an absorption spectrum and a fluorescence spectrum of the compound (E)-1;

FIG. 7 is a diagram showing an absorption spectrum and a fluorescence spectrum of the compound (Z)-1; and

FIG. 8 is a diagram showing electronic spectra of the respective compounds and a main transition of a CT absorption band of the (E)-1.

### BEST MODE FOR CARRYING OUT THE INVENTION

Hereinafter, it will be described with respect to exemplified embodiments of the present invention in detail with reference to the drawings.

### [Background for Reaching the Present Invention]

The present inventors have been promoting research sincerely with respect to construction of a complex system and expression of multiple property in which a photochromic compound that is a functional organic molecule and a transition metal complex & organic metal are coupled by π conjugation. For example, with respect to 3-ferrocenylazobenzene, research has been done with respect to trans→cis isomerization caused by excitation (green light) of charge transfer (CT) transition from ferrocene to azobenzene at a visible portion, a cis-trans switch depending on a single light utilizing a fact that the CT transition disappears caused by oxidization of the ferrocene, and the like.

Non-patent Document 1: J. Am. Chem. Soc. 2002, 124, 8800-8801
Non-patent Document 2: Angew. Chem., Int. Ed. 2006, 45, 4793-4795

Consequently, for the purpose of deeping the knowledge relating to the expression of the visible light isomerization caused by the charge transfer (CT) transition and the excitation thereof and at the same time, for the purpose of inducing material property control of a transition metal complex & organic metal by a photochromic compound (here, switch of electronic coupling strength in a mixed atomic valence state) concurrently with the expression of the visible light isomerization and achieving high functionalization of the complex system, the present inventors carried out evaluation of light-electrochemical material properties with respect to compounds (E)-2, (E)-10 mentioned below in which ferrocene is coupled with ethynyl then having Z-E optical isomerization capability by π conjugation and the like. Without being limited only by that, close inquiry was further carried out also with respect to (E)-1 in which there was employed substitution by triarylamine derivative which indicates an approximately equal redox behavior as ferrocene, and the like. Based on those facts, it became in a situation in which an unusual finding was obtained.

### [Material Properties]

Hereinafter, it will be explained with respect to the material properties of the compounds mentioned below. With respect to these compounds, it became clear in any one thereof that visible light isomerization and an electronic coupling switch associated therewith can be realized.

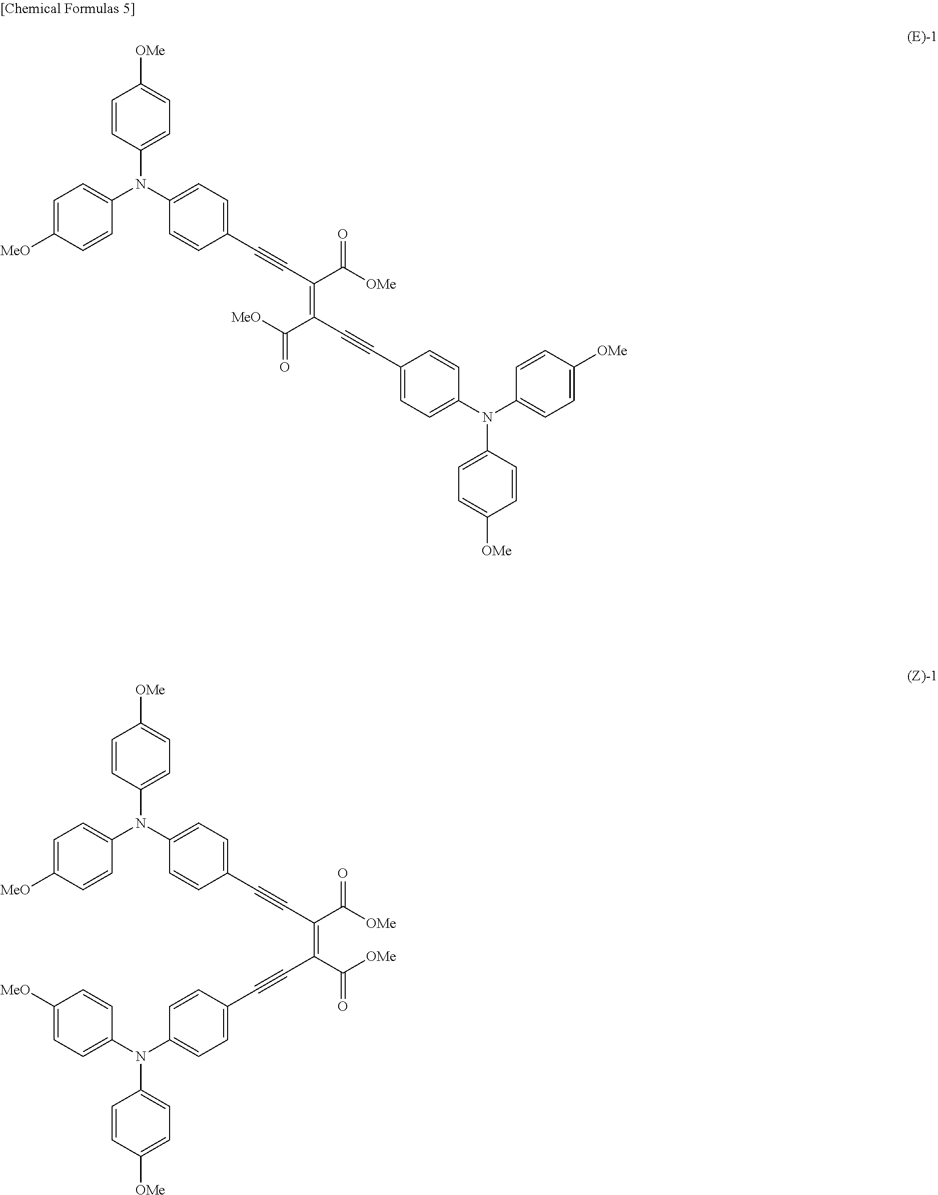

(E)-1: 2,3-bis(N,N-bis(p-anisyl)-4-aminophenylethynyl)dimethylfumarate
(Z)-1: 2,3-bis(N,N-bis(p-anisyl)-4-aminophenylethynyl)dimethylmaleate

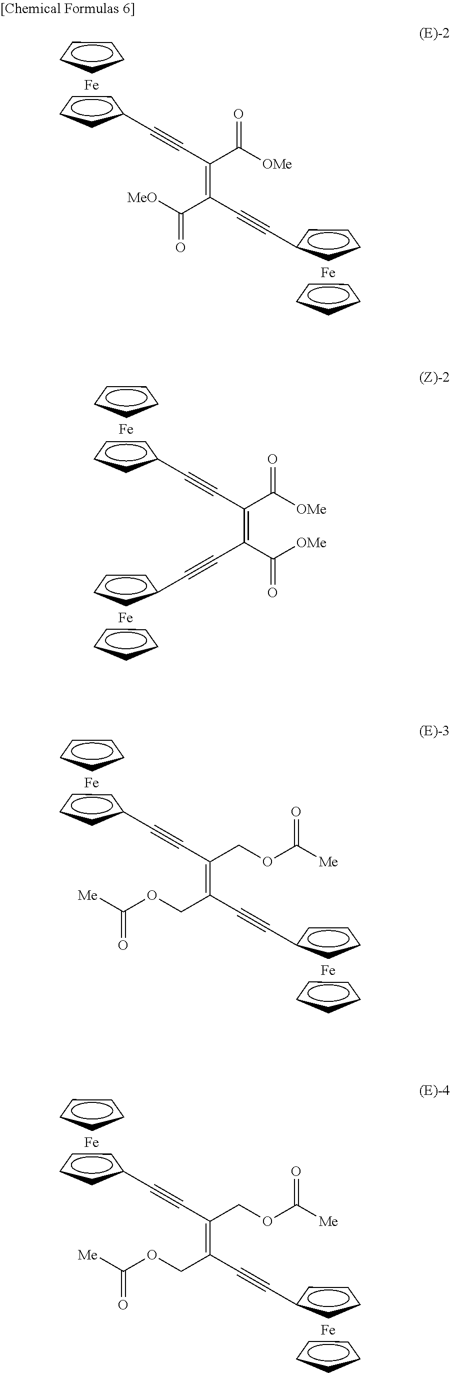

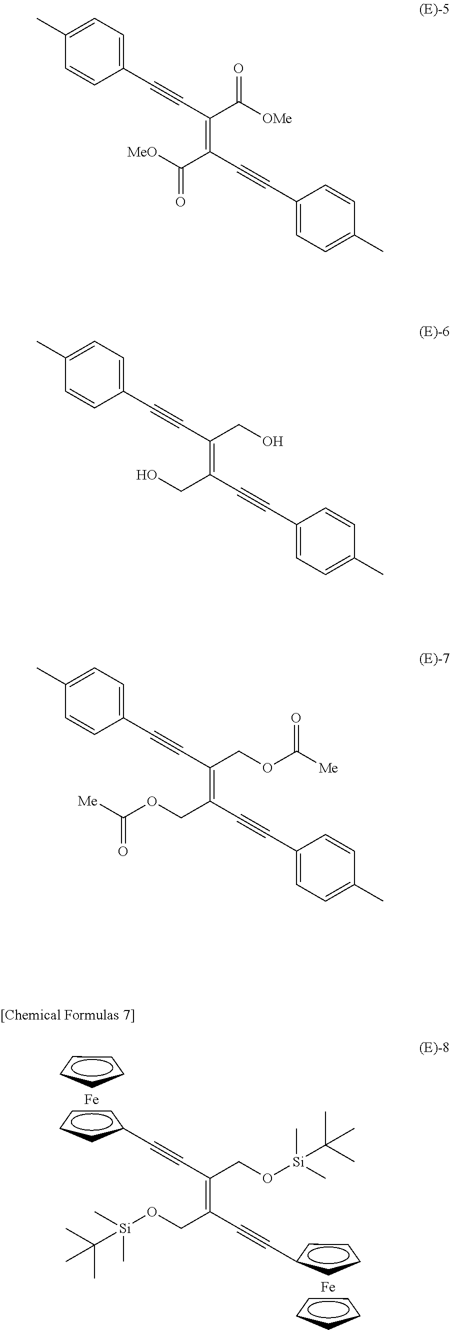

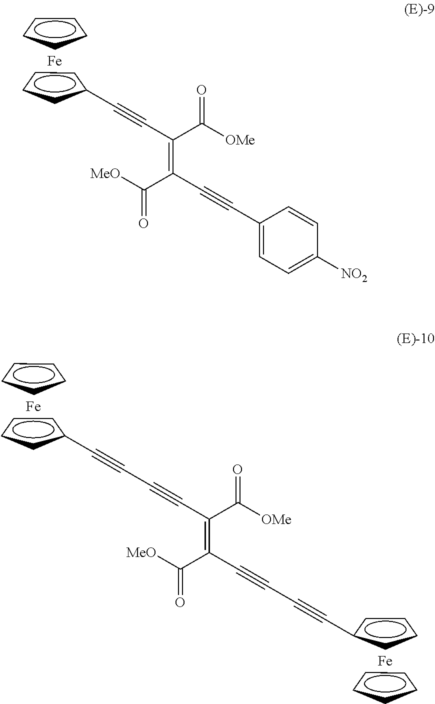

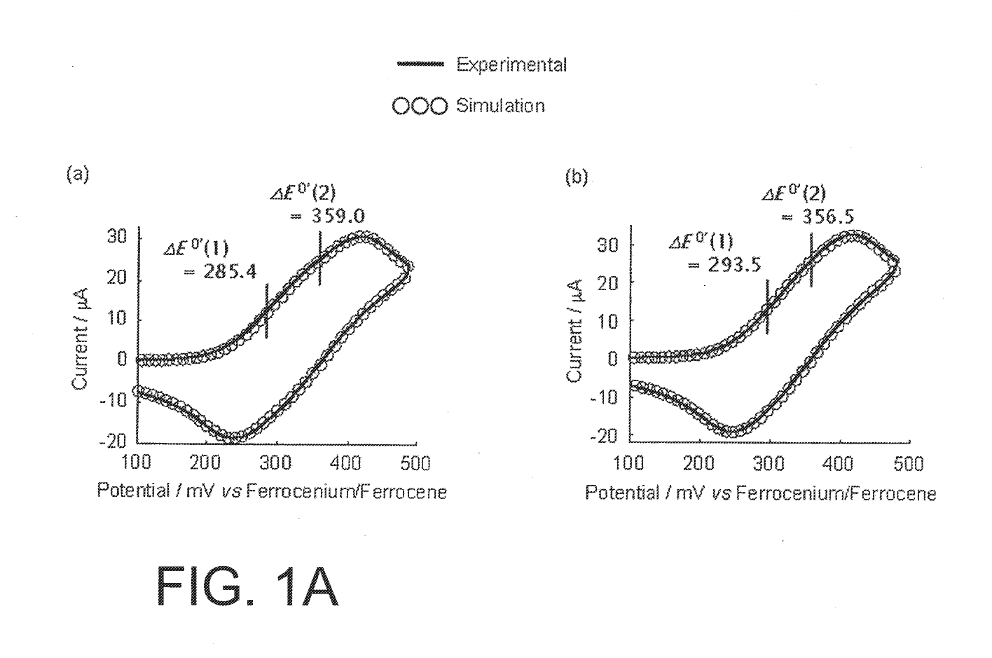

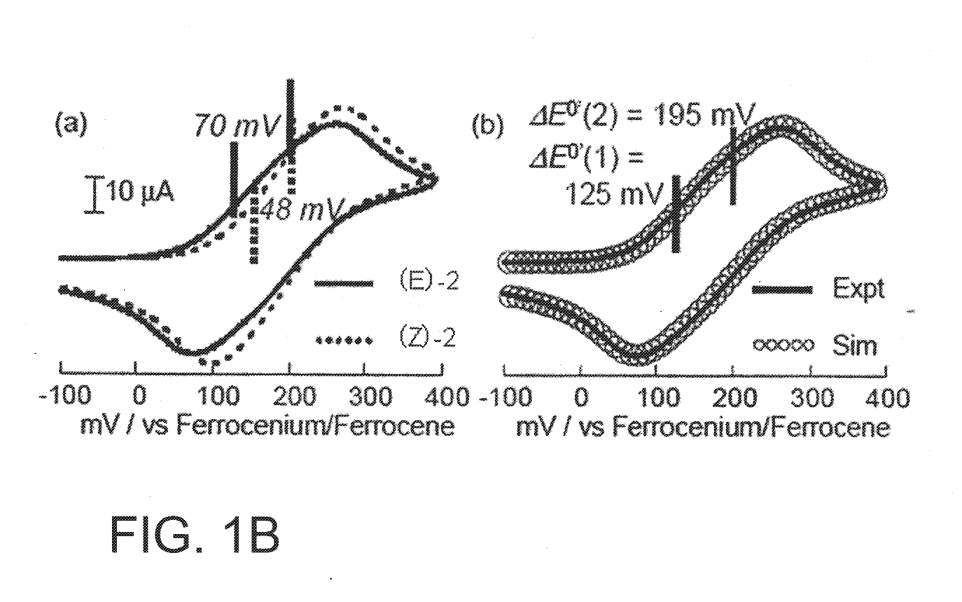

FIG. 1A is a diagram showing cyclic voltammograms of (a): a compound (E)-1 and (b): a compound (Z)-1, and simulations thereof. Measuring condition was such that sweep speed was 100 mVs⁻¹ in 1.0 mM, dichloromethane-0.1M n-tetrabutylammonium tetrafluoroborate and 3 mmφ glassy carbon was used as a working electrode. In (b), there is described also the standard electrode potential relating to one electron oxidation & reduction at the calculated triarylamine region. For the simulation, Digisim 3.03b (BAS Inc.) was used. From the drawing, it is comprehended that the compound is oxidized in two stages for every one electron, a hole transport capability is possessed, and the oxidation state is stable.

FIG. 1B is a diagram showing (a): cyclic voltammograms of (E)-2, (Z)-2 (measuring condition: 1.2 mM, dichloromethane-0.1M n-tetrabutylammonium-tetrafluoroborate, sweep speed of 100 mVs⁻¹, working electrode of 3 mmφGC), and (b): a simulation with respect to the voltammogram of (E)-2 and standard electrode potential relating to one electron oxidation & reduction at the obtained ferrocene region.

It will be considered about a switch of electronic coupling strength associated with the isomerization. As shown in the drawing, simulations of the cyclic voltammetry and the voltammogram were carried out and formula weight voltage difference ΔE^{0′} of the two ferrocene regions was calculated as 70 mV, 48 mV respectively for (E)-2, (Z)-2. A fact that larger ΔE^{0′} was presented for the E isomer although the spatial distance between Fe ions is much smaller for the Z isomer (in the crystal, (E)-2: 11.73 angstroms, (Z)-2: 6.17 angstroms) means that contribution of through-bondmutual action through an ethynylethen π conjugated chain is dominant with respect to the expression of the electronic coupling compared with through-spatial electrostatic reaction. In addition, also in the compound 1, there was observed a similar difference for the electronic coupling mutual action in the E, Z isomers. For both the compounds 1 and 2, distortion of the π conjugated system caused by a steric barrier of methylester group comrades in the Z isomer is suggested from the crystal structure and the electronic spectrum, and it is conceivable that this is a main cause of the lowering of the electronic coupling strength in the Z isomer.

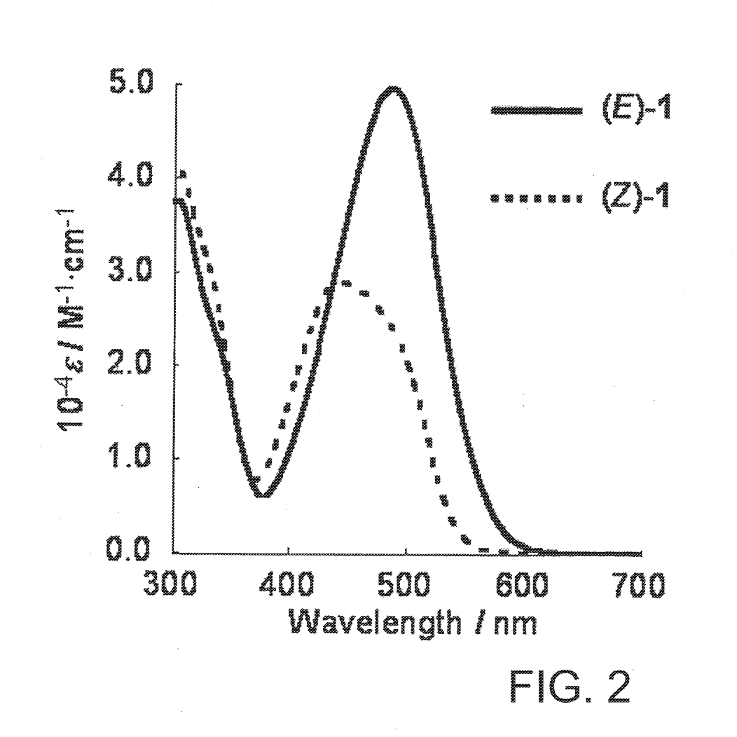

FIG. 2 is a diagram showing UV-vis is spectra of the compounds (E)-1, (Z)-1.

At the time of measurement, toluene solution was used.

Absorption of the visible region of the E isomer (400˜600 nm) is caused by one kind of charge transfer (CT) transition. While the allowance degree of the transition observed also in the E isomer lowers in the Z isomer along with the lowering of symmetry property, another CT transition which was under prohibition in the E isomer becomes allowable. Along with this, while the mole absorption constant decreases on the long wavelength side of the absorption of the visible region, that of the short wavelength side increases. This phenomenon is important in a reversible optical switch behavior between the Z-E isomers.

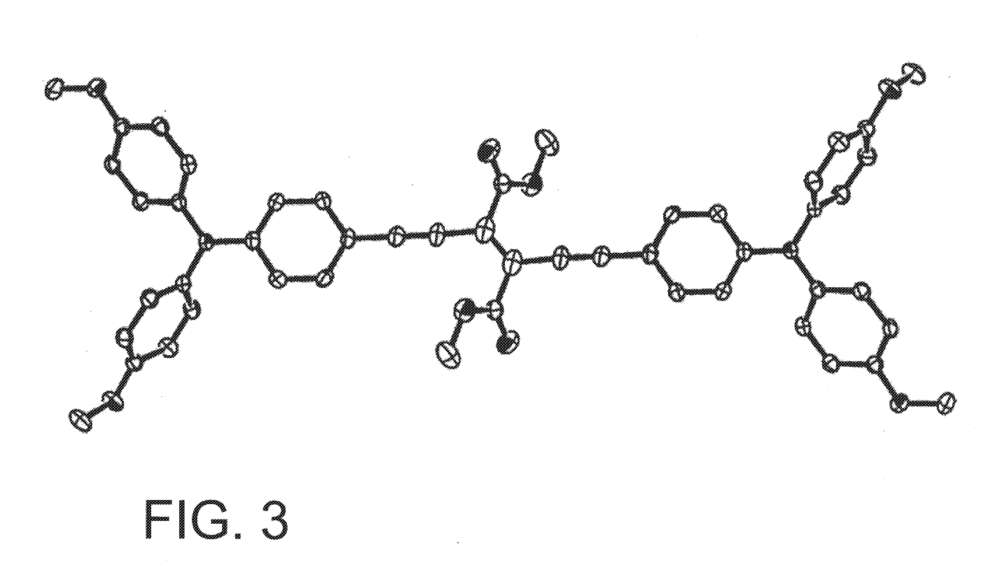

FIG. 3 is an ORTEP diagram of the compound (E)-1. Here, the hydrogen atom was omitted.

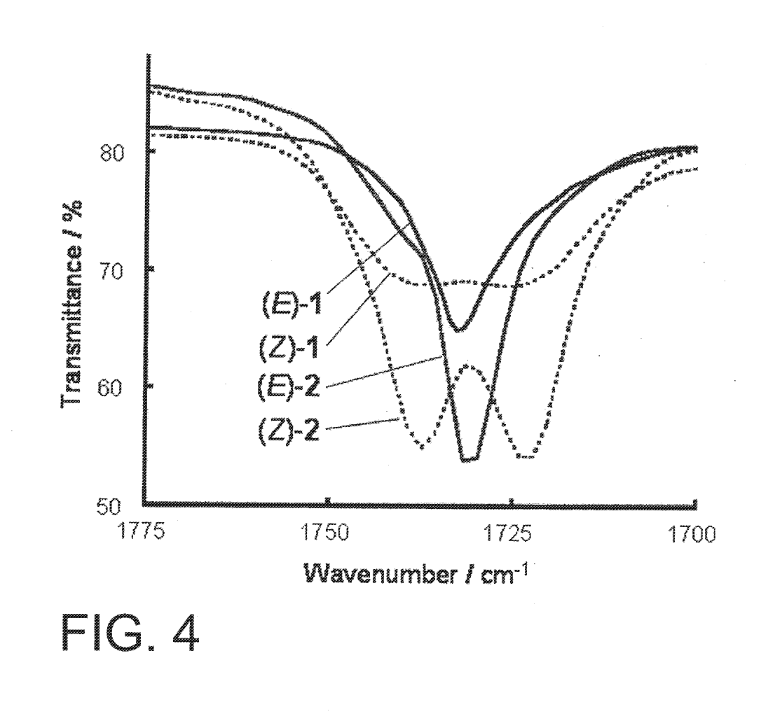

FIG. 4 is a drawing showing IR spectra originated from C═O stretching vibrations of the compounds (E)-1, (Z)-1, (E)-2 and (Z)-2. Other than the (Z)-1, the E, Z absolute conformations are determined by single crystal X-ray structure analyses. It is comprehended about Z isomer that the signal is divided along with the lowering of the molecule symmetry property.

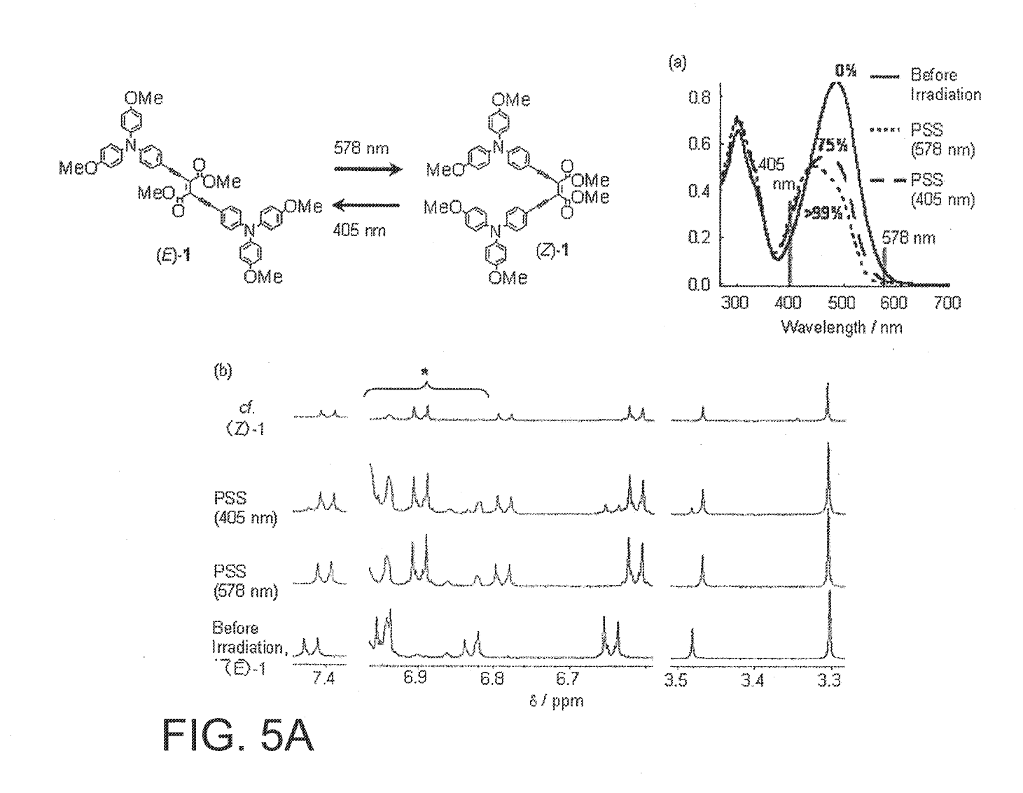

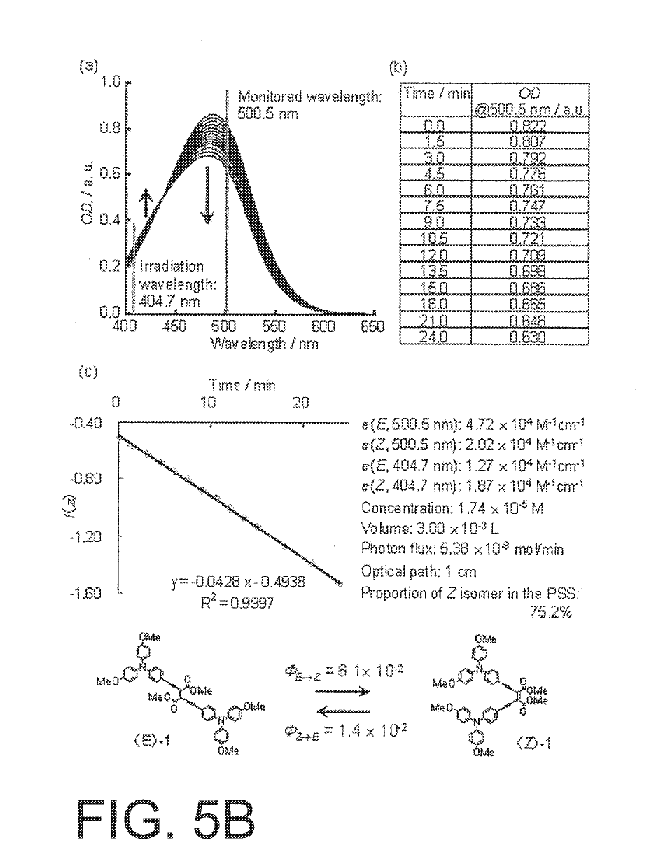

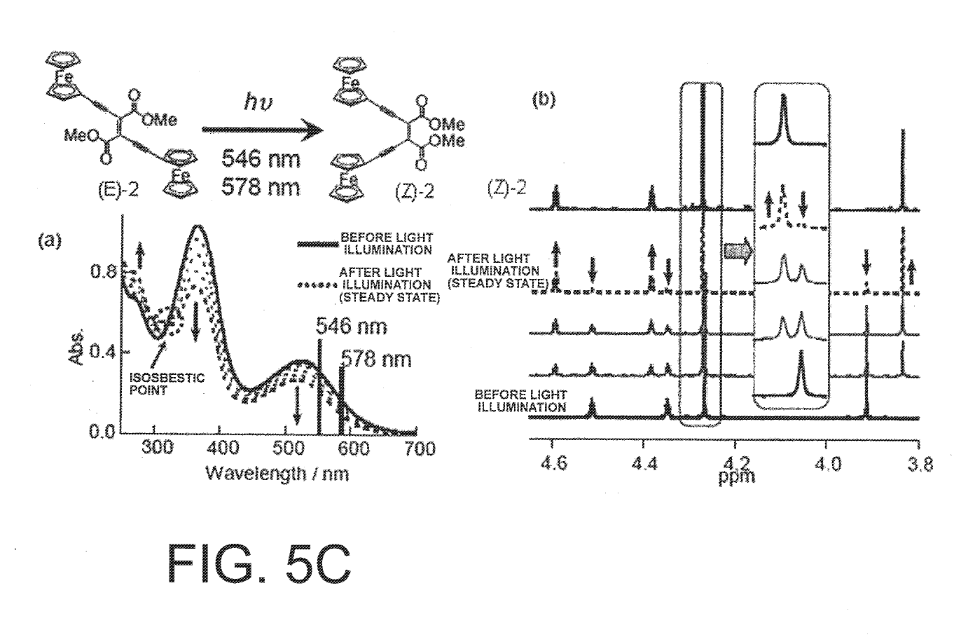

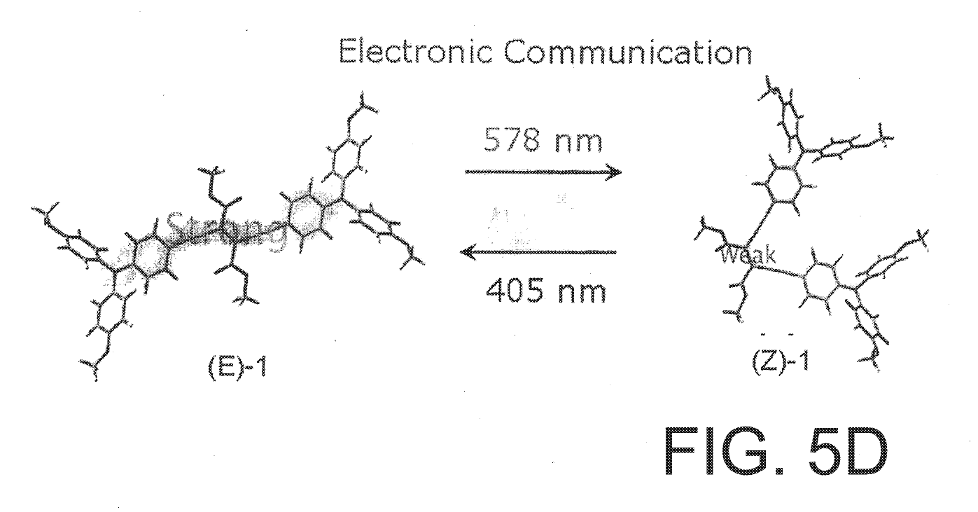

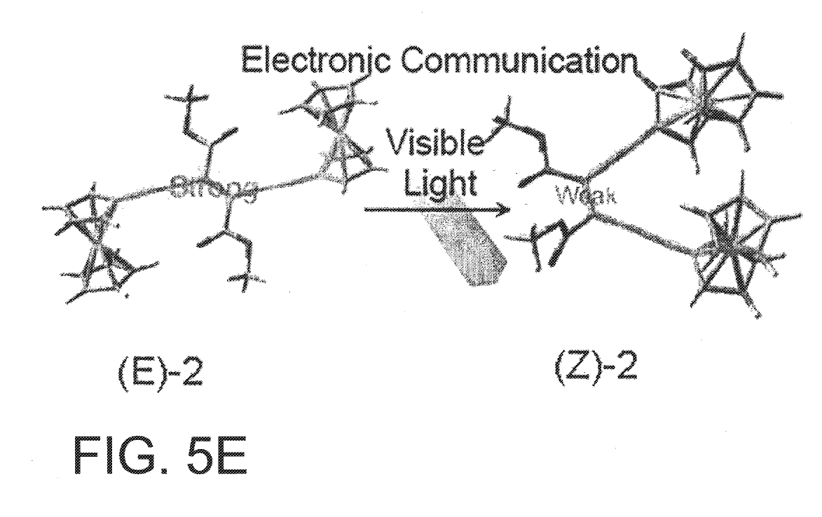

FIG. 5A is a diagram showing (a): a UV-vis spectral change (toluene solution) and (b): ¹H-NMR spectral change (toluene-d₈ solution) both associated with visible light illumination with respect to the compound (E)-1. In the drawing, percent (%) indicates the ratio of the Z isomer in a steady state of each light illumination. Also, “*” indicates a signal originated from deuterated solvent.

Here, it will be made reference to the isomerization behavior of the (E)-1. As shown in the drawing, there was presented isomerization to the Z isomer in toluene due to the excitation of the CT absorption band. In addition, it was also understood from the ¹H-NMR spectral change that the Z isomer ratio (when illuminating 578 nm) reaches approximately 100% in a photo steady state (PS). Further, it was found out that the Z isomer ratio in the PS decreases until 75% reversibly depending on the illumination of 405 nm visible light and that a molecule indicating Z-E optical switching ability is concerned. With respect to quantum yield, there was presented such a high value as Φ_{E→Z}=6.1×10⁻², Φ_{Z→E}=1.4×10⁻² (when illuminating 405 nm). FIG. 5B is a diagram showing (a) & (b): moment-to-moment change (in toluene) of the associated UV-vis spectrum with respect to the compound (E)-1 when illuminating a visible light (405 nm) thereon and (c): a plot of quantum-yield calculation and a used parameter. Here, there was used, for the mathematical processing, a method described in “Zimmerman, G.; Chow, Paik, U.-J. J. Am. Chem. Soc. 1958, 80, 3528-3531”.

FIG. 5C is a diagram showing (a): a UV-vis spectral change of the (E)-2 in the inside of CH₂Cl₂ and (b): a ¹H-NMR spectral change of the (E)-2 in the inside of CD₂Cl₂ & ¹H-NMR spectrum of the (Z)-2 both associated with a visible light (546 nm, 578 nm) illumination.

When excitation of the CT absorption band by 546 nm, 578 nm visible lights was executed with respect to the (E)-2, there was presented a stepwise decrease of the π-π*, CT absorption band together with one place of isosbestic point on the UV-vis spectrum ((a) in FIG. 5C), and there were observed, on the ¹H-NMR spectrum, a Z isomer production and an E isomer decrease associated therewith ((b) in FIG. 5C). The Z isomer existence ratio in the optical steady CT state (PS) was calculated as 89% from the integration ratio of the ¹H-NMR spectrum and also, the quantum yield (546 nm in toluene) was calculated as as Φ_{E→Z}=8.6×10⁻⁶, Φ_{Z→E}=2.5×10⁻⁶ from the moment-to-moment change of the UV-vis spectrum. These values are much smaller as compared with those of the compound 1.

With respect to the compound 2, there exists a rotational steric barrier between ferrocene comrades in the Z isomer. Also with respect to (E)-10 in which this steric barrier was avoided by extending the ethynyl group, 79% thereof was converted to the Z isomer in the PS by the excitation (578 nm) of the CT absorption band, but the quantum yield was Φ_{E→Z}=6.2×10⁻⁵, Φ_{E→Z}=3.4×10⁻⁵. A slight improvement was brought about, but it was comprehended that the steric barrier of the ferrocene comrades is not an essential factor of the low quantum yield.

FIG. 5D is a conceptional diagram between the triarylamines in the compound 1 and FIG. 5E is a conceptional diagram between the ferrocenes in the compound 2 respectively showing switches of electronic couplings by visible light illuminations. (E)-1, (E)-2 are unprecedented systems in which the electronic coupling strength between ferrocenes, between triarylamines and the like are switched respectively by the stimulation of the visible light. In particular, (E)-1 is excellent in an aspect that a reversible switch is possible.

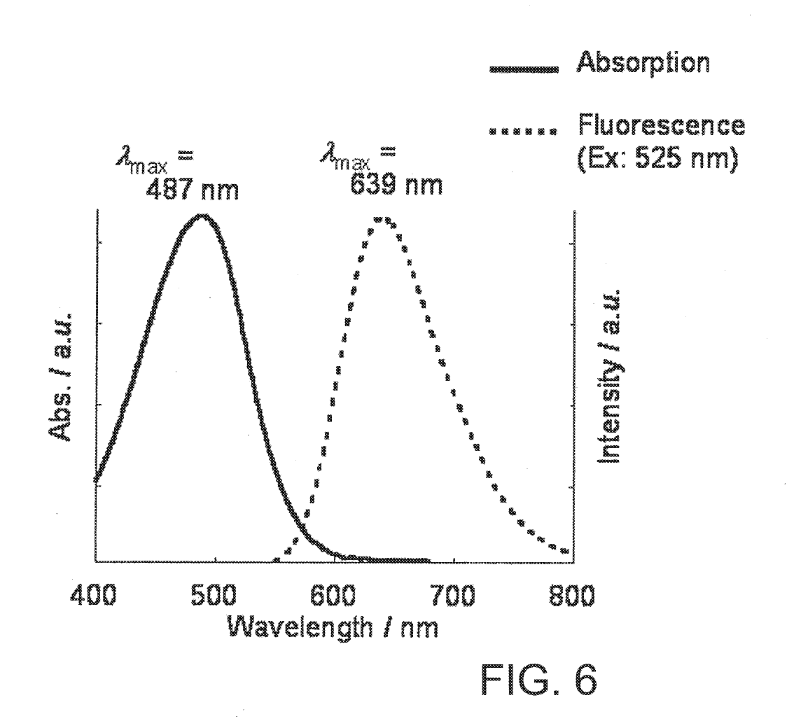

FIG. 6 is a diagram showing an absorption spectrum and a fluorescence spectrum of the compound (E)-1. The measurement was, carried out in toluene and at the room temperature.

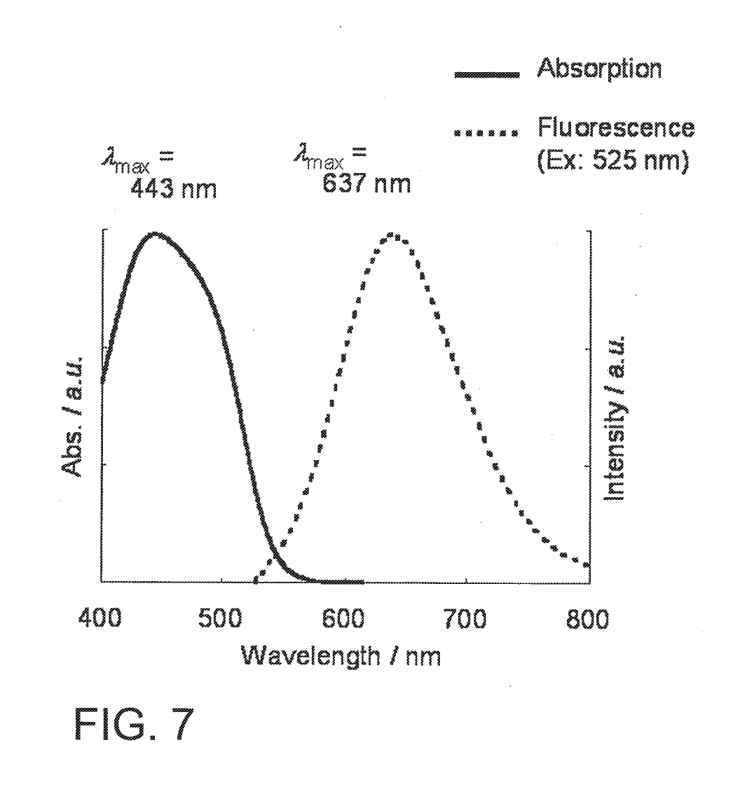

FIG. 7 is a diagram showing an absorption spectrum and a fluorescence spectrum of the compound (Z)-1. The measurement was carried out in toluene and at the room temperature.

It should be noted that it is confirmed that both the (E) isomer, (Z) isomer emit light in red even in the solid state when illuminating 365 nm ultraviolet lights or the like. In addition, it also became clear that the (E) isomer emits light more strongly than the (Z) isomer.

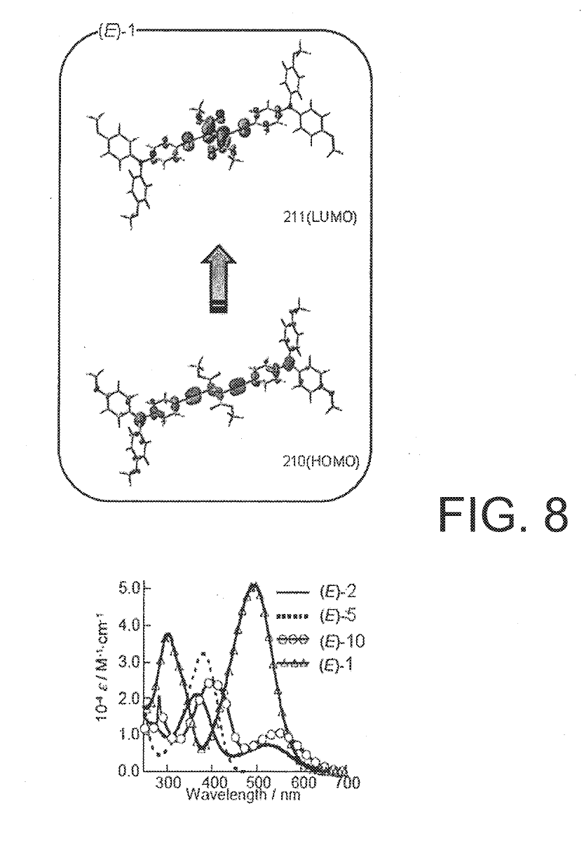

FIG. 8 is a diagram showing (E)-1, (E)-2, (E)-5, (E)-10 (in dichloromethane) and a main transition in an absorption band of a visible region (400-600 nm) of the (E)-1. Only a compound having a ferrocene or triarylamine region showed an absorption band at the visible portion. From the TDDFT calculation, it was attributed that the absorption band of those visible regions is a CT transition from an occupied orbital (HOMO) in which a ferrocened_{x2-y2} orbital or a triarylamine n orbital and an ethynylethen π orbital are conjugated to an ethynylethen π* orbital (LUMO).

It will be considered about the isomerization quantum yield. Both of ferrocene and triallylamine are compounds having nearly equal donor ability, but there exists a difference in a heavy atom effect and in ethynylethen existence or nonexistence of a ligand field (LF) excitation state having low energy. When considering about this difference, for the (E)-1, isomerization and fluorescence radiation advance efficiently from ¹CT which indicates a minimum singlet excitation state and on the other hand, for the (E)-2, internal conversion to ³LF occurs preferentially by way of intersystem crossing from ¹CT to ³CT or ³π-π*, so that it is conceivable that the isomerization thereof is disturbed. It should be noted that it is presently under review in (E)-2 about which excitation state within ¹CT, ³CT³ and ³π-π* directly gets involved in the isomerization.

According to the drawing, it is shown that the compound has a photochromic characteristic, in other words, has a possibility as an optical switch or an optical memory material. Also, from a fact that these compounds emit light, it is comprehended that they are rare compounds having a photochromic characteristic and a characteristic as a photoluminescent material. In addition, it also became clear that they have such an excellent characteristic in which even after repeating isomerization by light illumination, they are hardly dissolved by the light at all and are stable.

In addition, it is known also for the compound including N-arylcarbazol as mentioned below to be a molecule for which there are expected applications as an organic semiconductor & photoluminescent material. Also with respect to the introduction of N-arylcarbazol, it can be realized similarly as in a case of a derivative including triarylamine, which is shown hereinafter.

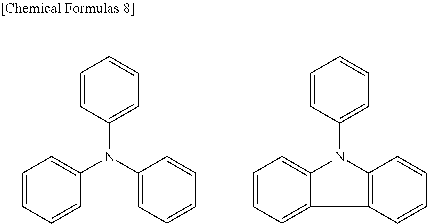

### Synthesis of Compound, etc.

### 2,3-BIS(N,N-BIS(P-ANISYL)-4-AMINOPHENYLETHYNYL)DIMETHYLFUMARATE

Under a nitrogen atmosphere, a mixture of 2,3-dibromo-dimethylfumarate (400 mg, 1.3 mmol), copper iodide (I) (27 mg, 0.14 mmol), bis(triphenylphosphine)palladium(II) dichloride (100 mg, 0.14 mmol), N,N-bis(p-anisyl)-4-ethynyl-benzenamine (940 mg, 2.8 mmol) and dehydrative triethylamine (40 mL) is heated & stirred for two hours at 100° C. After cooling, dichloromethane is added and insoluble components were removed by means of filtration through celite-cotton plug (celite is a registered trademark.) After evaporating the solvent under a reduced pressure, there was obtained a deep red colored solid of 2,3-bis(N,N-bis(p-anisyl)-4-aminophenylethynyl)dimethylfumarate by an amount of 770 mg (yield 73%) by refining the residue by using alumina column chromatography (activity II-III, hexane/dichloromethane=1/1). By recrystallizing the obtained solid from dichloromethane-hexane in a dark, place, a deep red colored crystal was obtained. ¹H-NMR (Toluene-d8): δ 7.42 (d, (9.2), 4H), 6.96-6.93 (m, 8H), 6.83 (d, (9.1), 4H), 6.64 (d, (9.6), 8H), 3.48 (s, 6H), 3.30 (s, 12H). Anal. Calcd for C₅₀H₄₂O₈N₂: C, 75.17; H, 5.30; N, 3.51. Found: C, 74.94; H, 5.37; N, 3.22.

### 2,3-BIS(N,N-BIS(P-ANISYL)-4-AMINOPHENYLETHYNYL)DIMETHYLMALEATE

The 2,3-bis(N,N-bis(p-anisyl)-4-aminophenylethynyl)dimethylfumarate (200 mg, 0.25 mmol) was dissolved into toluene (120 mL), and line spectra of 546-nm, 578-nm of a high pressure mercury lamp were illuminated for 24 hours. After evaporating the toluene under a reduced pressure, there was obtained a red colored powder of 2,3-bis(N,N-bis(p-anisyl)-4-aminophenylethynyl)dimethylmaleate by an amount of 185 mg (yield 92%) by refining the residue by using alumina column chromatography (activity II-III, hexane/dichloromethane=1/1). ¹H-NMR (Toluene-d8): δ 7.40 (d, (9.2), 4H), 6.90 (d, (9.5), 8H), 6.79 (d, (9.2), 4H), 6.61 (d, (9.6), 8H), 3.47 (s, 6H), 3.30 (s, 12H). Anal. Calcd for C₅₀H₄₂O₈N₂: C, 75.17; H, 5.30; N, 3.51. Found: C, 75.03; H, 5.55; N, 3.35.

### <(E)-2>

Under a nitrogen atmosphere, dehydrative triethylamine (60 mL) was added to 2,3-dibromo-dimethylfumarate (1.0 g, 3.3 mmol), copper iodide (I) (18 mg), bis(triphenylphosphine)palladium(II) dichloride (20 mg) and ethinyl ferrocene (1.5 g, 7.3 mmol). When heating a dark-brown colored suspension liquid at 100° C., it immediately changed to a red colored suspension liquid. Further, heating & refluxing were carried out for two hours and thereafter, dichloromethane was added after cooling, the obtained red colored suspension liquid was filtrated through celite-cotton plug, and insoluble materials were removed. The solvent was evaporated under a reduced pressure and by separating & refining the residue by using alumina column chromatography (activity II-III, hexane/dichloromethane=1/2), there was obtained a red colored solid of (E)-2 by an amount of 1.8 g (yield 98%). By recrystallizing the red colored solid from hexane-dichloromethane, a deep red colored crystal was obtained. ¹H-NMR (CD₂Cl₂): δ4.51 (dd, (2.0; 2.0), 4H), 4.35 (dd, (1.8, 1.8), 4H), 4.27 (s, 10H), 3.91 (s, 6H). Anal. Calcd for C₃₀H₂₄O₄Fe₂: C, 64.32; H, 4.32. Found: C, 64.04; H, 4.38.

### <(Z)-2>

(Z)-2 was obtained as a by-product of the (E)-2. The (Z)-2 by an amount of 98 mg was separated & refined from the (E)-2 by an amount of 5.0 g as a red colored solid depending on (activity II-III, hexane/dichloromethane=1/2). Further, by being recrystallized from dichloromethane-hexane, a red colored crystal was obtained. ¹H-NMR (CD₂Cl₂): δ 4.60 (dd, (1.8, 1.8), 4H), 4.38 (dd, (1.8, 1.8), 4H), 4.27 (s, 10H), 3.84 (s, 6H). Anal. Calcd for C₃₀H₂₄O₄Fe₂: C, 64.32; H, 4.32. Found: C, 64.15; H, 4.43.

### <(E)-3>

Under a nitrogen atmosphere, the (E)-2 (343 mg, 0.61 mmol) was dissolved into THF (40 mL) and the obtained deep red colored solution was cooled to −78° C. When a 1M toluene solution (3.1 mL, 3.1 mmol) of diisobutylaluminium hydride was dropped thereto and when the temperature was increased until a room temperature, it changed to an orange colored solution. After the situation was maintained and the stirring was continued for four hours and when methanol was added and the reaction was terminated, a white precipitate occurred. Filtration through celite-cotton plug was executed and the white precipitate was removed, and by evaporating the filtrated solvent, under a reduced pressure, there was obtained (E)-3 of an orange colored paste form by an amount of 238 mg (yield 77%). The further refinement was difficult, so that it was used for the next reaction without any change.

### <(E)-4>

Under a nitrogen atmosphere, the (E)-3 (238 mg, 0.47 mmol), N,N-dimethyl-4-aminopyridine (230 mg, 1.9 mmol) and 1-ethyl-3-(3-dimethylaminopropyl) carbodiimide hydrochloride (470 mg, 2.5 mmol) were dissolved into dehydrative dichloromethane (30 mL). Further, dehydrative triethylamine (0.32 mL, 4.4 mmol) and acetic acid (0.13 mL. 2.0 mmol) were added to an orange colored solution. Stirring was executed for seventeen hours and thereafter, water was added and the reaction was terminated.

Washing was carried out by water and saturated salt water and thereafter, the organic layer was dried by sodium sulfate, and after filtrating & separating the sodium sulfate, the solvent was evaporated under a reduced pressure. By separating & refining the residue by using alumina column chromatography (activity II-III hexane/dichloromethane=1/3), there was obtained an orange colored solid of (E)-4 by an amount of 56 mg (yield 20%). Further, by being recrystallized from dichloromethane-hexanean, there was obtained an orange colored needle shaped crystal. ¹H-NMR (CD₂Cl₂): δ4.96 (s, 4H), 4.47 (dd, (1.8, 1.8), 4H), 4.29 (dd, (1.8, 1.8), 4H), 4.23 (s, 10H), 2.15 (s, 6H). Anal. Calcd for C₃₂H₂₈O₄Fe₂: C, 65.34; H, 4.80. Found: C, 65.09; H, 4.82.

### <(E)-5>

Under a nitrogen atmosphere, dehydrative triethylamine (35 mL) was added to 2,3-dibromo-dimethylfumarate (1.3 g, 3.9 mmol), copper iodide (I) (20 mg), bis(triphenylphosphine)palladium(II) dichloride (13 mg) and p-tolylacetylene (1.0 mL, 7.9 mmol). When heating a colorless suspension liquid at 100° C., it immediately changed to a yellowish-brown colored suspension liquid. Further, heating & refluxing were carried out for four hours and thereafter, dichloromethane was added after cooling, and the obtained yellowish-brown colored suspension liquid was filtrated through celite-cotton plug and insoluble materials were removed. The solvent was evaporated under a reduced pressure, and by separating & refining the residue by using alumina column chromatography (activity II-III, hexane/dichloromethane=2/1), there was obtained a yellow colored solid of (E)-5 by an amount of 1.2 g (yield 82%). By recrystallizing the yellow colored solid from hexane-dichloromethane, a yellow colored crystal was obtained. ¹H-NMR (CD₂Cl₂): δ 7.40 (d, (8.0), 4H), 7.20 (d, (7.8), 4H), 3.91 (s, 6H), 2.38 (s, 6H). Anal. Calcd for C₂₄H₂₀O₄: C, 77.40; H, 5.41. Found: C, 77.31; H, 5.49.

### <(E)-6>

Under a nitrogen atmosphere, the (E)-2 (1.0 g, 2.7 mmol) was dissolved into THF (100 mL) and the obtained deep red colored solution was cooled to −78° C. A 1M toluene solution (14 mL, 14 mmol) of diisobutylaluminium hydride was dropped thereto and when it was ascending-heated until a room temperature, it was changed to a light brown colored solution. After the situation was maintained and the stirring was continued for two hours and when methanol was added and the reaction was terminated, a white precipitate occurred. Filtration through celite-cotton plug was carried out, the white precipitate was removed, the filtrate was evaporated under a reduced pressure and the solvent was removed. Separation & refinement were carried out to the residue by using alumina column chromatography (activity II-III, hexane/dichloromethane=1/3, and next, dichloromethane/methanol=50/1), and (E)-6 of a light yellow colored paste shape was obtained by an amount of 660 mg (yield 78%). The further refinement was difficult, so that it was used for the next reaction without any change.

### <(E)-7>

Under a nitrogen atmosphere, the (E)-3 (680 mg, 2.1 mmol), N,N-dimethyl-4-aminopyridine (1.0 g, 8.3 mmol) and 1-ethyl-3-(3-dimethylaminopropyl) carbodiimide hydrochloride (2.1 g, 11 mmol) were dissolved into dehydrative dichloromethane (30 mL). Further, dehydrative triethylamine (1.5 mL, 20 mmol) and acetic acid (0.62 mL. 9.5 mmol) were added to a colorless solution. Stirring was executed for nine hours and thereafter, water was added and the reaction was terminated. After washing was carried out by water and saturated salt water, the organic layer was dried by sodium sulfate and after filtrating & separating the sodium sulfate, the solvent was evaporated under a reduced pressure. By separating & refining the residue by using alumina column chromatography (activity II-III, hexane/dichloromethane=3/2), there was obtained a colorless solid (E)-7 by an amount of 370 mg (yield 44%). Further, by being recrystallized from dichloromethane-hexane, a colorless crystal was obtained. ¹H-NMR (CD₂Cl₂): δ7.36 (d, (8.0), 4H), 7.18 (d, (7.8), 4H), 5.03 (s, 4H), 2.37 (s, 6H), 2.12 (s, 6H). Anal. Calcd for C₂₆H₂₄O₄: C, 77.98; H, 6.04. Found: C, 77.90; H, 6.12.

### <(E)-8>

Under a nitrogen atmosphere, the (E)-4 obtained from the (E)-2 by an amount of 595 mg (1.1 mmol), N,N-dimethyl-4-aminopyridine (349 mg, 2.9 mmol) and di-tert-butylmethylsilyl chloride (642 mg, 4.3 mmol) were dissolved into dehydrative DMF (10 mL). This orange colored solution was added with dehydrative triethylamine (200 μl, 1.43 mmol) and was stirred for nineteen hours and thereafter, when methanol was added and the reaction was stopped, a white precipitate was occurred. Further, dichloromethane was added and thereafter, washing was carried out by water and saturated salt water, and the organic layer was dried by sodium sulfate and thereafter, the solvent was evaporated under a reduced pressure. By separating & refining the residue by using alumina column chromatography (activity II-III, hexane/dichloromethane=1/2), there was obtained an orange colored solid of (E)-8 by an amount of 284 mg (yield 36%, from (E)-2). By being recrystallized from dichloromethane, an orange colored crystal was obtained. ¹H-NMR (Toluene-d8): δ 4.71 (s, 4H), 4.43 (dd, (1.8, 1.8), 4H), 4.14 (s, 10H), 3.96 (dd, (1.8, 1.8), 4H), 1.08 (s, 18H), 0.26 (s, 12H). Anal. Calcd for C₄₀H₅₂Fe₂O₂Si₂: C, 65.57; H, 7.15. Found: C, 65.28; H, 7.19.

### <(E)-9>

Under a nitrogen atmosphere, dehydrative triethylamine (50 mL) was added to 2,3-dibromo-dimethylfumarate (443 mg, 1.5 mmol), copper iodide (I) (14 mg), bis(triphenylphosphine)palladium(II) dichloride (12 mg), 4-nitroethynylbenzene (210 mg, 1.4 mmol) and ethinyl ferrocene (300 mg, 1.4 mmol). When heating the obtained dark-brown colored suspension liquid to 100° C., it immediately changed to a red suspension liquid. Further, refluxing was carried out for two hours and thereafter, dichloromethane was added, filtration through celite-cotton plug was executed and insoluble components were removed. After evaporating the solvent under a reduced pressure, there was obtained a deep red colored solid of (E)-9 by an amount of 29 mg (4.1% yield) by refining the residue by using alumina column chromatography (activity II-III, hexane/dichloromethane=1/1). By recrystallizing the obtained solid from dichloromethane-hexane in a dark place, a deep red colored crystal was obtained. ¹H-NMR (Toluene-d8): δ 7.62 (d, (8.8), 2H), 4.38 (dd, (1.9, 1.9), 2H), 3.98-3.97 (m, 7H), 3.50 (s, 3H), 3.41 (s, 3H) (residual signal originated from proton was overlapped with that of toluene-d₈ and was not observed).

### <(E)-10>

Under a nitrogen atmosphere, 1,4-dioxane solution (100 mL, 1.0 mmol) of 1-ferrocenyl-1,3-butadiyne (148 mg, 0.49 mmol) was added to 2,3-dibromo-dimethylfumarate (148 mg, 0.49 mmol), copper iodide (I) (16 mg) and bis(triphenylphosphine)palladium(II) dichloride (16 mg). When dehydrative triethylamine (30 mL) was added to this brown colored suspension liquid and heating & refluxing at 100° C. was carried out for one hour, there was a change to a purple colored suspension liquid. After cooling, dichloromethane was added, filtration through celite-cotton plug was executed and insoluble materials were removed. The solvent was evaporated under a reduced pressure and by separating & refining the residue by using alumina column chromatography (activity II-III, hexane/dichloromethane=3/2), there was obtained a deep purple colored solid of (E)-7. Further, by recrystallizing the yellow colored solid from hexane-dichloromethane, a deep purple colored crystal was obtained by an amount of 54 mg (yield 18%). ¹H-NMR (Toluene-d8): δ 4.23 (dd, (1.8, 1.8), 4H), 3.91 (s, 10H), 3.86 (dd, (1.8, 1.8), 4H), 3.31 (s, 6H). Anal. Calcd for C₃₄H₂₄O₄Fe₂: C, 67.14; H, 3.98. Found: C, 66.98; H, 4.20.

### [Summary]

The molecule group of the compound mentioned above can become a high-performance light-electronic material. As a specific feature, for example, while maintaining high donor ability distinctive for a triarylamine class, the compound (E)-1 is formed as a dye which has a strong light absorbing force of around 50000M⁻¹ cm⁻¹ when converted to molar extinction coefficient ε. In addition, it possesses a high efficient photochromic characteristic in which the color changes by the visible light (quantum yield 6.1%), and both of the E isomer and the Z isomer (both the isomers) are concurrently thermally stable. Further, it presents fluorescence at a room temperature. Those features are important as a charge transport material, a photoelectric conversion material and a molecule optical memory & switch material.

### [Use Application]

It was possible to synthesize a novel high-performance formed electronic material by utilizing a fact that the triarylamine system molecule or the like has a strong electron donor ability. As a specific light-electronic function, it can be considered that it becomes a dye having a strong light absorbing force and it becomes a photochromic material & a photoluminescent material having a photochromic characteristic in which the color changes by the light.

Also, for example, a triarylamine derivative is practically used as a hole transport layer for an organic EL and is researched also as a high mobility material for an organic TFT, in addition, a fact that it is useful as a photoelectric conversion material for a solar battery by being incorporated in a charge transfer complex system has been researched. The molecule group in this exemplified embodiment has a photochromic characteristic, can switch an electron transport property by light, has strong light absorption capability and has also a charge transport property, so that it can become a photoelectron transport material and further, since it has an excellent characteristic by which it can become a photoluminescent material or the like, it is useful as a material for various kinds of organic light & electric devices.

It should be noted that since the compound mentioned above has an excellent rare characteristic, the following use applications are also conceivable. Those are use applications not only in the electronic field such as of a photoluminescent element, an optical recording material to be recorded with information due to color changing, an optical disc, a display of an electronic paper or the like, and a recording material & display material, but also, for such a decoration material to be light-emitted only during requirement, a photochromic ink, a sunglass, a goggle, a light shutter film, an optical filter, a display, a toy, an accessory, a paint, an ink, a curtain, a T-shirt, a swimsuit, fiber processing, an optical recording material, non-destructive read-out, a rewritable paper, an optical check card, and a sun checker or the like, and in addition, for a seal and a T shirt, for cosmetics of foundation check and the like, for a photochromic microcapsule which is excellent for the light resistance and which is for improvement of the speed of color-developing & color-erasing and for the like. Further, there can be also cited for application examples such as an ultra fine film structure, a super high density recording film, an optical memory, a biomimetic application of bacteriorhodopsin or the like, a surface nano pattern control, a recording medium of a single molecule optical memory or the like, a resin and a coloring agent for an eyeglass-lens to be colored, light-adjustment of a color rendering powder for skin or the like applied with photochromic technology, a photochromic coloring material, a rubbing free liquid crystal orientation film, a functional ink, an image display of a color display material or the like, a medicine discharge due to optical response viscoelasticity, an optical response complex formation, opening & closing of silicagel fine hole, a diffusion control of optical transformation or the like, all optical switches, a spatial light modulation material, an optical neural network system, an optical transformation control, a light control type photonic crystal, a fine pattern producing material, an optical & optoelectronic component of a polarizer or the like for optical communication, an organic & inorganic complex that is expanded & contracted caused by light, a fine optical drive device, a drive source of a micro machine, energy storage & conversion of direct conversion from optical energy to mechanical energy or the like, an optical antenna function, a forming control of DNA duplex and triplex, light control of fine hole alignment & fine hole diameter of a mesoporous silica thin film, normalization of an abnormal structure protein, reaction & structure & orientation control of an artificial DNA for the light control of protein synthesis or the like, thin film producing technique of sublimation diarylethene or the like, a high accuracy estimating system of light material property, material property observation & measurement and the like. From a fact that it strongly absorbs the UV-vis light of a wide range of wavelengths and the like, there can be also cited an application as a dye for a dye-sensitized solar battery as an example thereof.

### [Interpretation of Patent Right, etc.]

As mentioned above, it has been explained with respect to the present invention with reference to specific exemplified examples. However, it is just obvious that a person skilled in the art can achieve modification or substitution of the exemplified example within the scope thereof without departing from the gist of the present invention. More specifically, the present invention has been disclosed in a style of exemplification, so that the described contents of the present description must not be interpreted limitedly. In order to evaluate the gist of the present invention, the column of the claims described in the beginning should be taken into consideration.

Also, it is clear that the exemplified examples for the explanation of this invention will achieve the object mentioned above and on the other hand, it would be also understood that a person skilled in the art can employ a lot of modifications or other examples. It is also allowed to employ the element or the component in the scope of the claims, in the specification, in the drawings and in the respective exemplified examples together with another one or a combination thereof. The scope of the claims is intended to cover also such a modification and another exemplified example within that scope, and those are included within the technical idea and the technical scope of this invention.

### INDUSTRIAL APPLICABILITY

There are cited, as examples of use applications, such as a photochromic material, a photoluminescent material, a dye for a dye-sensitized solar battery and the like which have a photochromic characteristic in which color changes by light.

1. A compound, characterized by being expressed by a general formula (1) mentioned below or by a general formula (2) mentioned below. 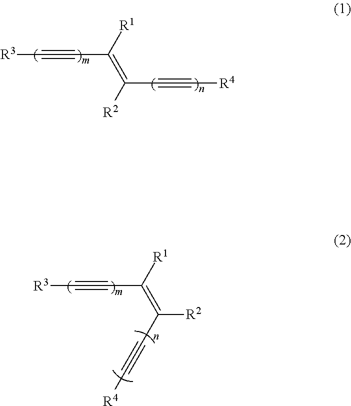
[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an 8-alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group; an aryloxymethyl group; an arylethynyl group; a 4-aryl-buta-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

2. The compound according to claim 1, characterized in that each of said R³ and said R⁴ is identical or different for every appearance thereof and is a group expressed by a general formula (3) mentioned below. 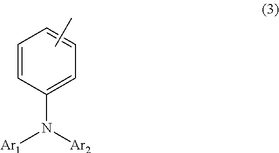
[In the formula, each of Ar¹ and Ar² is identical or different for every appearance thereof and indicates an aryl group having C atoms between 6 or more and 40 or less; a heteroaryl group having C atoms between 2 or more and 40 or less; an aryl group substituted by 1 or more R⁵ groups and having C atoms between 6 or more and 40 or less; or a heteroaryl group having C atoms between 2 or more and 40 or less. R⁵ is identical or different for every appearance thereof and indicates a linear, branched or annular alkyl group having H, F, Cl, Br, I, CN, NO₂, OH, Si(R⁶)₃, N(R⁶)₂, B(R⁶)₂ and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine); or an aryl group having C atoms between 6 or more and 40 or less; a heteroaryl group having C atoms between 2 or more and 40 or less; an aryloxy group having C atoms between 6 or more and 40 or less or a heteroaryloxy group having C atoms between 2 or more and 40 or less (it is allowed for each one of those groups to be substituted by a linear, branched or annular alkyl group having 1 or more of H, F, Cl, Br, I, CN, NO₂, OH, Si(R⁶)₃, N(R⁶)₂ and B(R⁶)₂, and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine). It is allowed for two or more R⁵ groups to mutually form monocyclic or polycyclic cyclic systems of fatty series or aromatic series. R⁶ is identical or different for every appearance thereof and indicates an aliphatic or aromatic hydrocarbon group having H and C atoms between 1 or more and 20 or less.]

3. The compound according to claim 1, characterized in that each of said R³ and said R⁴ is identical or different for every appearance thereof and is a group expressed by a general formula (4) mentioned below. 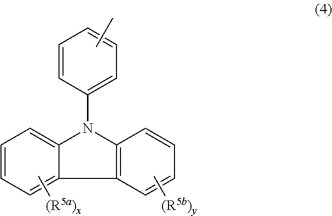
[In the formula, each of x and y is an integer value between 0 or more and 4 or less. Each of R^{5a} and R^{5b} is identical or different for every appearance thereof and indicates a linear, branched or annular alkyl group having H, F, Cl, Br, I, CN, NO₂, OH, Si(R⁶)₃, N(R⁶)₂, B(R⁶)₂ and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine); an aryl group having C atoms between 6 or more and 40 or less; a heteroaryl group having C atoms between 2 or more and 40 or less; an aryloxy group having C atoms between 6 or more and 40 or less; or a heteroaryloxy group having C atoms between 2 or more and 40 or less (it is allowed for each one of those groups to be substituted by a linear, branched or annular alkyl group having 1 or more of H, F, Cl, Br, I, CN, NO₂, OH, Si(R⁶)₃, N(R⁶)₂ and B(R⁶)₂, and C atoms between 1 or more and 40 or less; an alkoxy group or a thioalkoxy group (here, it is allowed for 1 or more noncontiguous C atoms to be substituted by —CR⁶═CR⁶—, —C≡C—, NR⁶—, —O—, —S—, —CO—O— or —O—CO—O— and further, it is allowed for 1 or more H atoms to be substituted by fluorine). It is allowed to form a monocyclic or polycyclic cyclic system of fatty series or aromatic series among two or more of R^{5a} group comrades or R^{5b}) group comrades, or between a R^{5a} group and a R^{5b} group. R⁶ is identical or different for every appearance thereof and indicates an aliphatic or aromatic hydrocarbon group having H and C atoms between 1 or more and 20 or less.]

4. A compound, characterized by being expressed by a formula (5) mentioned below or by a formula (6) mentioned below.

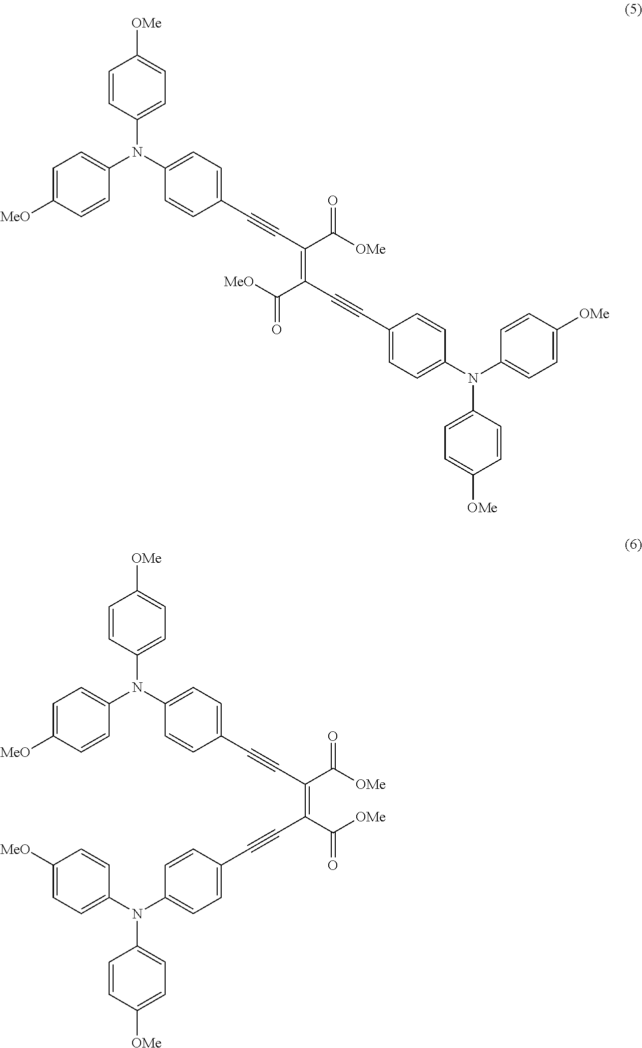

5. A photochromic material, characterized by using the compound according to claim 1 as the material thereof.

6. An electronic material, characterized by using the compound according to claim 1 as the material thereof.

7. A compound producing method, characterized by producing a compound expressed by a general formula (2) mentioned below by illuminating an electromagnetic wave onto a compound expressed by a general formula (1) mentioned below. 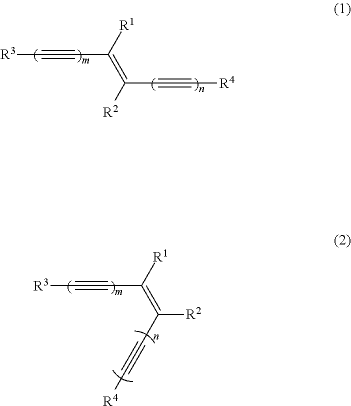
[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an 8-alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group an aryloxymethyl group; an arylethynyl group; a 4-aryl-buta-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

8. A compound producing method, characterized by producing a compound expressed by a general formula (1) mentioned below by illuminating an electromagnetic wave onto a compound expressed by a general formula (2) mentioned below. 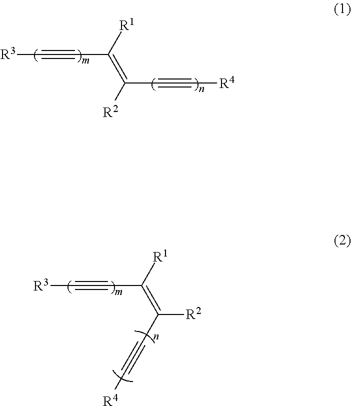
[In the formula, each of R¹ and R² is identical or different for every appearance thereof and indicates an ethynyl group; a buta-1,3-dienyl group; a hexa-1,3,5-triynyl group or an octa-1,3,5,7-tetrynyl group (here, it is allowed for the terminal hydrogen atom to be substituted by a carboxy group, a cyano group, a sulfo group or a phospho group); or a linear, branched or annular alkyl group having C atoms between 1 or more and 40 or less; an alkylester group; an alkoxymethyl group; an alkylsiloxymethyl group; an alkylethynyl group; a 4-alkyl-buta-1,3-dienyl group; a 6-alkyl-hexa-1,3,5-triynyl group or an 8-alkyl-octa-1,3,5,7-tetrynyl group (here, it is allowed for the alkyl group to be substituted by 1 or more fluorines, a carboxy group, a cyano group, a sulfo group or a phospho group); or an aryl group having C atoms between 6 or more and 40 or less; an arylester group; an arylsiloxymethyl group; an aryloxymethyl group; an arylethynyl group; a 4-aryl-buta-1,3-dienyl group; a 6-aryl-hexa-1,3,5-triynyl group or an 8-aryl-octa-1,3,5,7-tetrynyl group (here, it is allowed for the aryl group to be substituted by 1 or more fluorines, a carboxy group, a cyano group, a sulfo group or a phospho group); or a heteroaryl group having C atoms between 2 or more and 40 or less; a heteroarylester group; a heteroarylsiloxymethyl group; a heteroaryloxymethyl group; a heteroarylethynyl group; a 4-heteroaryl-buta-1,3-dienyl group; a 6-heteroaryl-hexa-1,3,5-triynyl group or an 8-heteroaryl-octa-1,3,5,7-tetrynyl group (here, it is allowed for the heteroaryl group to be substituted by 1 or more fluorines, a carboxy group, a cyano group, a sulfo group or a phospho group); or an acetoxymethyl group, a fluoroacetoxymethyl group, a hydroxylmethyl group, a carboxy group, a cyano group, a sulfo group or a phospho group. Each of R³ and R⁴ is identical or different for every appearance thereof and indicates a ferrocenyl group; an N,N-diaryl-4-aminophenyl group; an N,N-diaryl-3-aminophenyl group; an N,N-diaryl-2-aminophenyl group; a 4-(carbazol-9-yl)phenyl group; a 3-(carbazol-9-yl)phenyl group; a 2-(carbazol-9-yl)phenyl group or a derivative of one of those groups. Respective m and n are integer values of 1 or more and it is allowed for them to be identical or different.]

9. A 2,3-bis(N,N-bis(p-anisyl)-4-aminophenylethynyl)dimethylfumarate producing method, characterized in that a 2,3-dibromo-dimethyl fumarate and an N,N-bis(p-anisyl)-4-ethynyl-benzenamin are reacted.

10. A 2,3-bis(N,N-bis(p-anisyl)-4-aminophenylethynyl)dimethylfumarate producing method, characterized in that light is illuminated onto a 2,3-bis(N,N-bis(p-anisyl)-4-aminophenylethynyl)dimethylmaleate.

11. A 2,3-bis(N,N-bis(p-anisyl)-4-aminophenylethynyl)dimethylmaleate producing method, characterized in that light is illuminated onto a 2,3-bis(N,N-bis(p-anisyl)-4-aminophenylethynyl)dimethylfumarate.
